(19) United States
(12) Patent Application Publication
(10) Pub. No.: US 2023/0225972 A1
(43) Pub. Date: Jul. 20, 2023

(54) LIPOSOMAL COMPOSITION FOR PREVENTING OR EARLY TREATMENT OF PATHOGENIC INFECTION

(71) Applicant: STATENS SERUM INSTITUT, Copenhagen S (DK)

(72) Inventors: Frank FOLLMANN, Copenhagen S (DK); Peter ANDERSEN, Copenhagen S (DK); Jes DIETRICH, Copenhagen S (DK); Dennis CHRISTENSEN, Copenhagen S (DK)

(73) Assignee: STATENS SERUM INSTITUT, Copenhagen S (DK)

(21) Appl. No.: 17/996,379

(22) PCT Filed: Apr. 15, 2021
(86) PCT No.: PCT/EP2021/059807

§ 371 Date: Oct. 17, 2022

(30) Foreign Application Priority Data
Apr. 15, 2020 (EP) 20169618.4
Oct. 12, 2020 (EP) 20201268.8

(51) Int. Cl. A61K 9/127; A61K 47/18; A61K 47/10; A61K 31/713; A61P 31/04; A61K 9/00

(52) U.S. Cl. CPC A61K 9/1271; A61K 47/186; A61K 47/10; A61K 31/713; A61P 31/04; A61K 9/0043; A61K 9/007

(57) ABSTRACT

The present invention relates to a liposomal composition for use as a medicament. In particular, the present invention relates to a liposomal composition for use in prevention or early treatment of pathogenic infection. More specifically, the liposomal composition is used for prevention, or early treatment, of pathogenic infection in the respiratory tract, preferably by nasal or pulmonary administration.

### TECHNICAL FIELD OF THE INVENTION

The present invention relates to a liposomal composition for use as a medicament. In particular, the present invention relates to a liposomal composition for use in prevention or early treatment of pathogenic infection. More specifically, the liposomal composition is used for prevention or early treatment of pathogenic infection in the respiratory tract, preferably by nasal or pulmonary administration.

### BACKGROUND OF THE INVENTION

The innate immune system is designed to act within moments of encountering emerging pathogens. By strengthening the innate immune response in the airways a fast protection against infection with airway pathogens can be achieved. This will be particular relevant for individuals with compromised/reduced immunity in the airways. Unlike an adaptive immune response, activation of innate immunity will give broad protection against many pathogens, and will not be compromised by mutational changes in the pathogen.

By strengthening this immune response in the airways, in particular in high risk groups with reduced airway immunity, lives can be saved. One particular example is the corona virus SARS-CoV-2 causing the CoVid-19 disease. Coronaviruses use several methods to evade the innate immune system, and even compromise it, leading to infection and a subsequent less effective adaptive immunityl. It is therefore crucial to prevent this immune evasion, by strengthening the innate first line of defense. SARS-CoV-2 shows great resemblance to SARS-CoV-1. Studies with SARS-CoV-1 infection have clearly demonstrated the importance of innate immunity. In fact, even in the complete absence of T and B cells, animals were able to control the infection. This control required the presence of innate immunity, with the ability to produce various cytokines and in particular type I interferons^{2,3}. In line with this, other studies showed that both prophylactic and post-exposure strategies involving only innate immune stimulation was able to prevent infections in animal models¹. A key component of the host defense against virus infections is the Toll-Like Receptor-3 (TLR-3), which is activated by dsRNA from viruses and triggers the production of type I interferons⁴. Interestingly, treatment of mice with TLR3 agonists through the respiratory tract reduced pulmonary virus titers caused by airway infection with five different strains of influenza^{5,6}. In a mouse model of SARS, based on a mouse adapted highly virulent strain of SARS Corona virus, a formulation of TLR3 agonist gave impressive levels of antiviral activity. This formulation called Ampligen has recently entered human clinical trials for intravenous administration¹¹. In humans, a phase I/II trial administration of a TLR3 agonist also protected against airway infection with both rhinovirus and influenza virus⁷. Thus, TLR3 stimulation is a very promising strategy to boost innate anti-viral immunity.

Another key component for airway protection against pathogens is the C-type lectin receptor Mincle, which has been associated with innate protection against e.g. Mycobacteria and Streptococcus. The inventors have furthermore shown that the combination of a mincle agonist and TLR3 synergistically increases the ability to induce the type 1 interferon dependent CD8 T cell responses.

The overall hypothesis behind the present invention is that prophylactic intranasal or pulmonary treatment with a liposomal composition according to the present invention will activate the innate immune system, including the mucosal immune response, and lead to a short term protection against pathogenic infection, such as SARS-CoV-2 infection, as well as other respiratory pathogens. This treatment can be given repeatedly throughout an epidemic to high-risk populations, and significantly impact morbidity and inhibit the dissemination of e.g. SARS-CoV-2. Furthermore, this treatment is not compromised by any mutations in the pathogen, since it is non-specific.

Statens Serum Institut has developed several vaccine adjuvants/innate immune activators based on the CAF® (Cationic Adjuvant Formulation) technology, one of which is CAF®09b that is currently being tested in humans. It is being tested as an adjuvant given either intramuscularly or intraperitoneally with the purpose of inducing a T and B cell mediated adaptive immune response against vaccine antigens (Clinical trials's ongoing: NCT03412786; NCT03715985, NCT03926728). CAF®09b contains the innate immunostimulator MMG (binding to the innate receptor, Mincle), and the type-I interferon inducing TLR3 agonist poly I:C. As a parenteral adjuvant CAF®09b results in strong innate immune activation, but no severe side effects⁸.

Another member of the CAF® family is CAF®04, which induces Th1 activation through release of IFN-γ. The present invention focus on administration of liposomal compositions, such as CAF®04 or CAF®09b, administered to the airways with the purpose of activating airway innate immune cells to protect against an airway infection with pathogens.

### SUMMARY OF THE INVENTION

Intranasal treatment with a liposomal composition, such as CAF®04 or CAF®09b, activate the innate immune system, including the mucosal immune response, and lead to a short term protection against infection with respiratory pathogens. This treatment can be given repeatedly throughout an epidemic, e.g. to high-risk populations, and significantly impact morbidity and the dissemination of the pathogen.

Thus, an object of the present invention relates to a liposomal composition comprising the cationic lipid dimethyldioctadecylammonium (DDA) and at least one immunomodulator for use as a medicament in a subject.

Another aspect of the present invention relates to a liposomal composition comprising the cationic lipid dimethyldioctadecylammonium (DDA) and at least one immunomodulator for use in the prevention or treatment of pathogenic infection of the respiratory tract in a subject.

The liposomal composition may also comprise an immunomodulator, preferably poly (I:C) and more preferably STING agonists, such as cAMP, cGMP, c-di-AMP or c-di-GMP.

The pathogenic infection may be a virus infection of the respiratory tract caused by a virus that may be selected from but not limited to picornavirus, rhinovirus, coronavirus, such as MERS-corona virus, SARS-corona virus, such as SARS-CoV-2, influenza virus, human parainfluenza virus, human respiratory syncytial virus, adenovirus, enterovirus, and metapneumovirus.

In another embodiment of the present invention, the pathogenic infection is a bacterial infection of the upper respiratory tract caused by bacteria that may be selected from but not limited to Chlamydia pneumoniae, Streptococcus pneumoniae, Streptococcus pyrogenes, Haemophilus influenza, Moraxella catarrhalis and a mycobacterium, such as M. tuberculosis, M. bovis, M. africanum, M. canetti, M. microti., and Burkholderia Sp.

Pathogenic infections caused by virus and bacteria of the respiratory system can be particularly serious in elderly and weak patients and patients with chronic or congenital dysfunction of the respiratory system, such as but not limited to asthma, cystic fibrosis, or chronic obstructive pulmonary disease (COPD).

Accordingly, the subject, who will benefit especially from the present invention, may have chronic obstructive pulmonary disease (COPD), asthma cystic fibrosis, or another condition that results in compromised respiratory function compared to a healthy subject.

Yet another aspect of the present invention is to provide a device for nasal administration comprising the liposomal composition as disclosed herein.

### BRIEF DESCRIPTION OF THE FIGURES

FIG. 1 illustrates intranasal treatment with the liposomal composition, CAF®09b (comprising DDA, MMG and Poly (I:C)), which activates the innate immune system and lead to a short term protection against infection with respiratory pathogens. This treatment can be given repeatedly throughout an epidemic to high-risk populations and significantly reduce morbidity and the dissemination of the pathogen.

FIG. 2 illustrates the influx of innate immune cells and the secretion of IFN-alpha/beta in the lung of mice +/−CAF®09b treatment.

FIG. 3 illustrates the gene expression level of selected proinflammatory and type 1-interferon markers in the lung of mice +/−CAF®09b treatment.

FIG. 4 illustrates the gene expression level of selected proinflammatory and type 1-interferon markers in the lung of mice with no treatment or mice treated with either two or four times with CAF®09b.

FIG. 5 illustrates the development over time of weight and survival in mice +/−CAF®09b treatment and subsequently infected with influenza virus.

FIG. 6 illustrates the development of IgG antibody in the blood of the mice in FIG. 5.

FIG. 7 illustrates the development over time of survival, viral load and weight in mice +/−CAF®04 and STING agonist alone or in combination.

FIG. 8 illustrates the development over time of survival, viral load and weight in mice +/−CAF®09b and STING agonist alone or in combination.

FIG. 9 illustrates the development of viral replication in the nose of mice +/−CAF®04, CAF®09b and STING agonist alone or in combination.

FIG. 10 illustrates the development of weight in hamsters infected with SARS-CoV-2, +/−CAF®09B or Mock control.

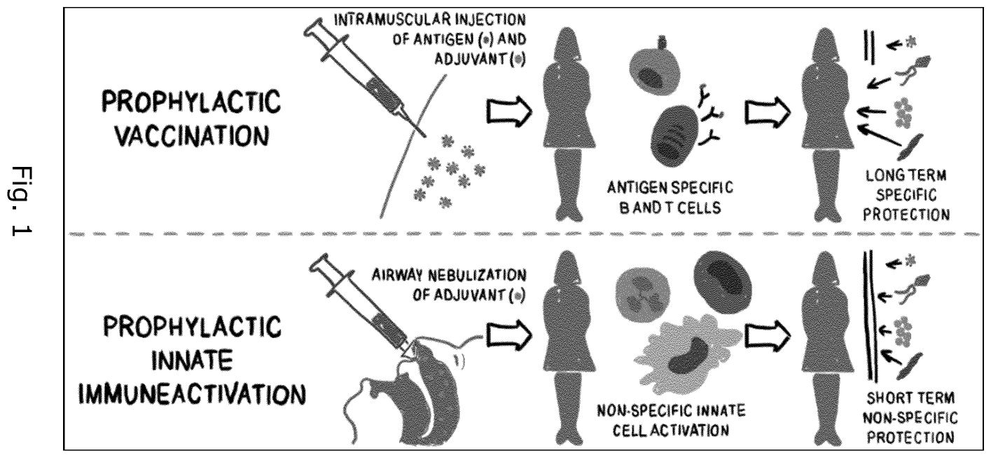

The present invention will now be described in more detail in the following.

### DETAILED DESCRIPTION OF THE INVENTION

### Definitions

Prior to discussing the present invention in further details, the following terms and conventions will first be defined:

### Subject

The term “subject” comprises humans of all ages, other primates (e.g., cynomolgus monkeys, rhesus monkeys); mammals in general, including commercially relevant mammals such as cattle, pigs, horses, sheep, goats, cats, and/or dogs; and/or birds. Preferred subjects are humans.

The term “subject” also includes healthy subjects of the population and, in particular, healthy subjects, who are exposed to pathogens and in need of protection against infection, such as health personal.

Pathogenic infections caused by virus and bacteria of the respiratory system can be particularly serious in elderly and weak patients and patients with chronic or congenital dysfunction of the respiratory system, such as asthma, cystic fibrosis, or chronic obstructive pulmonary disease (COPD).

Patients with lung cancer may also receive the liposomal composition according to the present invention. Smokers, with either or both of a past history of smoking or an ongoing use of cigarettes or other smoking products.

The abovementioned subjects are vulnerable to upper respiratory infections, so administration of a disclosed composition could potentially prevent upcoming common cold symptoms or illnesses and thus prevent exacerbations of their underlying illnesses and symptoms.

### Administration

The term “administration” in the context of the present invention means administration in various modes either by systemic administration, such as injection, in delivery systems, by topical administration, intradermal administration, (intra)nasal administration, sublingual or pulmonary administration.

The liposomal composition according to the present invention is typically administered by (intra)nasal administration in the range of once per day, to once or twice per week, to once per two weeks, to once or twice per month.

When administered nasally or intranasally, the terms “administered nasally”, “nasal administration” or “intranasal administration” are used interchangeably and refer to a delivery of the liposomal composition to the mucosa of the subject's nose such that the liposomal composition content is absorbed directly into the nasal tissue or upper respiratory tract.

The liposomal composition according to the present invention can also be administered by pulmonary administration. The term “pulmonary” refers to an administration through the subject's nose or mouth to deliver the liposomal composition to alveolar lung tissues where it is absorbed into the body. The pulmonary administration may be direct inhalation of the liposomal composition, such as a powder, or inhalation of an aerosol that contains the composition. The liposomal composition according to the present invention contemplated herein may be administered by joint nasal and pulmonary administration where a portion of the liposomal composition is delivered to the nasal mucosa and a portion is delivered to the alveolar lung tissues.

### Infections

The term “infection” in the context of the present invention means an infection in the respiratory tract, such as the upper or lower respiratory tract, caused by a pathogen, such as a virus or bacteria.

The viral infection may be a human coronavirus infection or an influenza virus infection. Other viral infections may be caused by a picornavirus (e.g., rhinovirus), human parainfluenza virus, human respiratory syncytial virus, adenovirus, enterovirus, or metapneumovirus.

The bacterial infection of the respiratory tract may be caused by a bacteria selected from the group selected from Chlamydia pneumoniae, Streptococcus pneumoniae, Streptococcus pyrogenes, Haemophilus influenza, Moraxella catarrhalis and a mycobacterium, such as M. tuberculosis, M. bovis, M. africanum, M. canetti, M. microti., and Burkholderia Sp.

The “prevention” of a pathogenic infection, condition or disease refers to a liposomal composition that, in a statistical sample, reduces the occurrence of the infection, condition or disease in the treated subject relative to an untreated subject, or delays the onset or reduces the severity of one or more symptoms of the infection, condition or disease relative to the untreated control subject.

### DDA

One particular effective type of adjuvant that promotes a cell-mediated immune response is the class of quaternary ammonium compounds, such as the cationic surfactant dimethyldioctadecylammonium (DDA). DDA is a synthetic amphiphilic compound comprising a hydrophilic positively charged dimethylammounium head group and two long hydrophobic alkyl chains. In an aqueous environment, DDA molecules self-assemble to form vesicular bilayers similar to liposomes made from natural phospholipids.

Thus, DDA is a synthetic surfactant consisting of a hydrophilic, cationic quaternary ammonium headgroup, and two hydrophobic saturated C18 alkyl chains. Due to their surfactant properties, DDA molecules self-assemble into liposome-like structures upon dispersion in aqueous media.

The liposomal composition according to the present invention comprises the cationic lipid DDA as various salts, most preferably dimethyldioctadecylammonium bromide or chloride (DDA-B or DDA-C) or the sulfate, phosphate or acetate salt hereof (DDA-X), or dimethyldioctadecenylammonium bromide or chloride (DODA-B or DODA-C) or the sulfate, phosphate or acetate compound hereof (DODA-X). Most preferably, the liposomal composition according to the present invention comprises dimethyldioctadecylammonium bromide.

The CAS number of DDA is 3700-67-2.

However, the liposomal composition according to the present invention can comprise further cationic lipids.

### MMG

Mycobacterial lipid monomycoloyl glycerol (MMG) is a glycolipid, which stabilizes the liposome formed with cationic surfactant DDA by incorporation into the liposome membrane.

The cationic liposomes are stabilized by incorporating glycolipids, such as MMG and optionally further glycolipids, into the liposome membranes.

Glycolipids like MMG have immunostimulatory properties themselves and can act in a synergistic way with the quaternary ammonium compounds (DDA) to enhance the immune response.

The synthetic analogue, referred to as MMG-1, consists of a hydrophilic glycerol headgroup and a lipid acid, displaying two hydrophobic saturated C14/C15 alkyl tails, linked via an ester bond. Furthermore, an array of MMG analogues, differing in the alkyl chain lengths (MMG-2; C16/C17, MMG-3; C10/C11, and MMG-4; C6/C7), or with respect to stereochemistry of headgroup (MMG-5; 2S) and lipid tail (MMG-6) exists.

MMG is preferably the synthetically manufactured glycolipid, MMG-1.

The chemical structure of the preferred MMG analogue is 3-hydroxy-2-tetradecyl-octadecanoic acid-2,3-dihydroxypropyl ester, preferably the (2R)-2,3-Dihydroxypropyl-3-hydroxy-2-tetradecyloctadecanoate diastereomer.

### Poly (I:C)

The term “Poly (I:C)” or “Poly I:C” according to the present invention comprises single-stranded polyinosinic acid (Poly I) and single-stranded polycytidylic acid (Poly C) that are not associated by hydrogen bonding or covalent bonding at the time of administration as well as double-stranded or complexed Poly I/Poly C. Upon administration to a moist mucosal surface, uncomplexed Poly I and Poly C can form complexed Poly(I:C) and thus prime the innate immune system and provide protection against viral infection.

Preferably, Poly (I:C) is a synthetically manufactured double-stranded RNA analogue consisting of strands of polyinosinic acid annealed to strands of polycytidilic acid or analogues thereof. poly (A:U) (Polyadenylic-polyuridylic acid) could be used as an alternative analogue.

The molecular weight of Poly (I:C) depends on the polymer length. The Poly I:C potassium salt has a molecular weight specification of 10-750 kDa with a preferred range of 100-750 kDa. The CAS number of Poly I:C is 24939-03-5.

### Cyclic Dinucleotide

The term “cyclic dinucleotide” according to the present invention describes a group of compounds including c-di-GMP, c-di-AMP, 3′,3′-cyclic-GMP-AMP (3′,3′cGAMP) and 2′,3′-cyclic-GMP-AMP (2′,3′cGAMP), which work as potent immunostimulatory molecules or immunomodulators. The second messenger c-di-GMP, structured as a cycle containing two guanine bases linked by ribose and phosphate, has been shown to activate “stimulator of interferon genes” (STING), i.e. STING agonists, resulting in an increased IFN-I secretion.

### Further Adjuvants

In the present context, the term “adjuvant” refers to a compound or mixture that further enhances the immune response. An adjuvant can serve as a tissue depot that slowly releases the antigen and as a lymphoid system activator, which non-specifically enhances the immune response, i.e. an immunomodulator.

By an immunomodulator is meant any component, which increases the effect of or acts synergistically to obtain an immune response. This potentiation could be done unspecifically or specifically through pattern recognition receptors (PRRs) including but not limited to C-type lectin receptors (CLRs), RIG-like receptors (RLRs). nucleotide-binding oligomerization domain (NOD) proteins and the toll-like receptors (TLRs).

The immunogenicity of the liposomes can be potentiated by inclusion of immunostimulating ligands (a.k.a. immunomodulators) for the so-called pattern recognition receptors (PRRs) recognizing conserved molecular structures known as pathogen-associated molecular patterns (PAMPs) on pathogens. The ability of the PAMP to modulate the innate immune response, and thereby the ensuing adaptive response, can with advantage be exploited for use in the prevention or treatment of pathogenic infection of the respiratory tract.

The so-called pattern recognition receptors (PRRs) include C-type lectin receptors, RIG-like receptors, nucleotide-binding oligomerization domain (NOD) proteins, and the ever-growing toll-like receptor (TLR) family. The PAMPs vary among the pathogens and include molecules such as cord factor (TDM), flagellin, lipopolysaccharide (LPS), peptidoglycans, and several nucleic acid variants, such as double-stranded ribonucleic acids (dsRNAs). Many immunomodulators inspired by the PAMPS have been developed over the years. These include, trehalose dibehenate (TDB), synthetic monomycolyl glycerol (MMG), monophosphoryl lipid A (MPL), polyinosinic acid:polycytidylic acid (poly(I:C)). The combination of liposomes with these PAMPs/immunomodulators is an attractive approach to develop a way of preventing or treating early pathogen infection, where the PAMPs/immunomodulators stimulate the antigen presenting cells, thereby potentiating the immune response.

Further adjuvants include, but are not limited to Quil A, QS21, aluminium hydroxide, Freund's incomplete adjuvant, monophosphoryl lipid A (MPL), Trehalose Dimycolate (TDM), Muramyl Dipeptide (MDP), “IC31”, saponin, surface active substances, such as lysolecithin, pluronic polyols, polyanions, peptides, oil or hydrocarbon emulsions, keyhole limpet hemocyanins, dinitrophenol or combinations hereof. Preferably, the adjuvant is pharmaceutically acceptable.

### Liposome

The term “liposome” or “liposomal composition” is a broad definition for vesicles composed of lipid bilayers enclosing aqueous compartments. The membrane-forming lipids are amphiphilic and accordingly contain a polar and an apolar region.

The polar region typically consist of a phosphate group, an acidic group and/or tertiary or quaternary ammonium salts and can either have a net negative (anionic), neutral or positive (cationic) surface charge at physiological pH, depending on the composition of the lipid head groups. The pH is preferably adjusted to physiological pH such as by dispersion adjusted to pH 5.0-8.0 in Tris or histidine buffer, most preferably adjusted to pH 6.5-7.5. The apolar region typically consists of one or more fatty acid chains with at least 8 carbons and/or cholesterol. The lipids constituting the vesicular bilayer membranes are organized such that the apolar hydrocarbon “tails” are oriented toward the center of the bilayer while the polar “heads” orient towards the in- and outside aqueous phase, respectively.

Thus, “liposome” or “liposomal” is defined as closed vesicle structures made up of one or more lipid bilayers surrounding an aqueous core. Each lipid bilayer is composed of two lipid monolayers, each of which has a hydrophobic “tail” region and a hydrophilic polar “head” region. In the lipid bilayer, the hydrophobic “tails” of the lipid monolayers orient toward the inside of the bilayer, while the hydrophilic “heads” orient toward the outside of the bilayer. Liposomes can have a variety of physicochemical properties such as size, lipid composition, surface charge, fluidity and number of bilayer membranes. According to the number of lipid bilayers, liposomes can be categorized as unilamellar vesicles (UV) or small unilamellar vesicles (SUV) comprising a single lipid bilayer or multilamellar vesicles (MLV) comprising two or more concentric bilayers each separated from the next by a layer of water. Water soluble compounds are entrapped within the aqueous phases/core of the liposomes opposed to lipophilic compounds, which are trapped in the core/center of the lipid bilayer membranes.

The term “cationic lipid” or “cationic liposome” is intended to include any amphiphilic lipid, including natural as well as synthetic lipids and lipid analogs, having hydrophobic and polar head group moieties, a net positive charge at physiologically acceptable pH, and which can form bilayer vesicles or micelles in water.

### Other Cationic Lipids as Further Adjuvants

Further cationic lipid compounds, which may be incorporated in the liposomal composition according to the invention as further adjuvants, are 1,2-dioleoyl-3-trimethylammonium propane (DOTAP), 1, 2-dimyristoyl-3-trimethylammonium-propane, 1,2-dipalmitoyl-3-trimethylammonium-propane, 1,2-distearoyl-3-trimethylammonium-propane, dioleoyl-3-dimethylammonium propane (DODAP), N-[1-(2,3-dioleyloxy)propyl]-N,N,N-trimethylammonium (DOTMA), octadecenoyloxy(ethyl-2-heptadecenyl-3-hydroxyethyl) imidazolinium (DOTIM) 1,2-dioleyl-sn-glycero-3-ethylphosphocoline (DOEPC) and 3-tetradecylamino-tert-butyl-N-tetradecylpropion-amidine (diC14-amidine).

Fatty acid chain refers to a branched or unbranched saturated or unsaturated hydrocarbon chain of alkyl or acyl groups.

The term pharmaceutically acceptable refers to a substance, which does not interfere with the effectiveness or the biological activity of the active ingredients and which is not toxic to the host or the patient.

The cationic liposomes are stabilized by incorporating glycolipids, such as MMG and optionally further glycolipids, into the liposome membranes. By incorporating is meant procedures to imbed a molecule's hydrophobic region and hydrophilic region in a corresponding hydrophobic and hydrophilic region or moiety of a membrane, micelle, liposome or bilayer. Procedures for incorporating glycolipids in liposomes can be “the thin film method”, “the reverse-phase evaporation method”, “the organic solution injection method”, the “double emulsion method”, the “High-shear mixing method”, the “microfluidics method” and future—at the present time unknown methods having the same effect of incorporating glycolipids into the liposome membranes. The presently known methods are mentioned in the background of invention chapter.

A glycolipid is defined as any compound containing one or more monosaccharide or glycerol residues bound by a glycosidic linkage to a hydrophobic moiety, such as a long chain fatty acid, acylglycerol, a sphingoid, a ceramide or a prenyl phosphate. The further glycolipids of this invention can be of synthetic, plant or microbial origin e.g. from mycobacteria.

One class of glycolipids, which may be used in this invention in addition to MMG as a further adjuvant, is acylated (or alkylated) glycosides, which consist of one or two sugars residues esterified to one, two or even three fatty acids. The fatty acids can be either straight chain including saturated fatty acids e.g. myristic acid C14:0, pentadecanoic acid C:15, palmitic acid C16:0, heptadecanoic acid C17:0, steric acid C18:0, nonadecanoic acid C:19, arachidic acid, C:20, heneicosanoic C21:0, behenic acid C:22 and unsaturated fatty acids e.g. oleic acid C18:1n-9 linoleic acid 18:2n-6, or complex branched fatty acids such as mycolic acid, methoxymycolic acids, ketomycolic acids, epoxymycolic acids and corynomycolic acid. The sugar residues can be either simple monosaccharides, e.g. glucose and fructose, or disaccharides comprising two covalently linked monosaccharides, e.g. sucrose consisting of glucose and fructose and trehalose, in which two glucose units are joined by a glycosidic linkage. One type of glycolipids used in this invention is cell wall glycolipids isolated from mycobacterium, which consist of a disaccharide esterified to one, two, or three either normal palmitic acid, C16:0; oleic acid, C18:1n-9; linoleic acid, 18:2n-6 or complex hydroxy, branched-chain fatty acids i.e. mycolic acid residues ranging in length from 60 to 90 carbon atoms. Other bacterial glycolipids used in this invention have shorter fatty acid chains e.g. corynomycolic (22-36 carbons) or nocardomycolic (44-60 carbons) acids isolated from Corynobacterium, Nocardia. A preferred mycobacterial glycolipid is alpha,alpha′-trehalose 6,6′-dimycolate (TDM) often referred to as cord factor, which is one of the most important immunomodulatory components of the mycobacterial cell wall. In a particular preferred embodiment the glycolipid consist of the disaccharide alpha,alpha′-trehalose esterified to two docosanoic fatty acids (behenic acid) e.g. alpha,alpha′-trehalose 6,6′- dibehenate (TDB), which is a pure synthetic analog to TDM. Another preferred mycobacterial glycolipid is of course monomycolyl glycerol or preferably the synthetic analog 3-hydroxy-2-tetradecyl-octadecanoic acid-2,3-dihydroxypropyl ester or closely related compounds with shorter or longer acyl backbones.

Other classes of glycolipids, which may be used in this invention, include but are not limited to:

Glycolipids based on glycerol: These lipids consist of a mono- or oligosaccharide moiety linked glycosidically to the hydroxyl group of glycerol, which may be acylated (or alkylated) with one or two fatty acids. Furthermore, these glycolipids may be uncharged and, therefore often called neutral glycoglycerolipids, or may contain a sulfate or a phosphate group. A preferred glycolipid of this class according to the invention is monomycolyl glycerol (MMG) (Andersen et al 2009a, Andersen et al 2009b) or synthetic analogs hereof, such as 3-hydroxy-2-tetradecyl-octadecanoic acid-2,3-dihydroxypropyl ester or closely related compounds with shorter or longer acyl backbones (Andersen et al 2009b, Nordly et al 2011).

Further glycolipids based on ceramides: Glycosphingolipids have according to the structure of the carbohydrate moiety been divided into neutral glycosphingolipids containing an unsubstituted glycosyl group and acidic glycosphingolipids containing a glycosyl group with an acidic carboxyl, sulphate, or phosphate group.

Lipopolysaccharides (LPS) may be used as a further adjuvant. These complex compounds are the endotoxic antigens found in the cell walls of Gram-negative bacteria (S-lipopolysaccharides). The lipid part (Lipid A) forms a complex with a polysaccharide through a glycosidic linkage. Lipid A consists of a backbone of b-1,6-glucosaminyl-glucosamine with two phosphoester groups in the 1-position of glucosamine I and in the 4-position of glucosamine II. The 3-position of glucosamine II forms the acid-labile glycosidic linkage to the long-chain polysaccharide. The other groups are substituted (in Escherichia) with hydroxylated fatty acids as hydroxymyristate (two ester-linked and two amide-linked) and normal fatty acids (laurate). A particular preferred lipopolysaccharide of this invention is monophosphoryl derivatives of lipid A (MPL), which are non toxic and have excellent adjuvant properties.

Glycosides of sterols may be used as a further adjuvant. This family consists of one carbohydrate unit linked to the hydroxyl group of one sterol molecule. The sterol moiety was determined to be composed of various sterols: cholesterol, campesterol, stigmasterol, sitosterol, brassicasterol and dihydrositosterol. The sugar moiety is composed of glucose, xylose and even arabinose.

Glycosides of fatty acids or alcohols may be used as a further adjuvant. A great number of simple glycolipids are found in bacteria, yeasts and in lower organisms (sponges). These compounds are composed of a glycosyl moiety (one or several units) linked to one hydroxyl group of a fatty alcohol or a hydroxy fatty acid or to one carboxyl group of a fatty acid (ester linkage). These compounds frequently possess interesting physical or biological properties. Some of them are industrially produced for their detergent properties (alkyl glycosides).

Furthermore, macromolecules, e.g. oligonucleotides, peptide or protein antigens, can be entrapped within the aqueous phase of both unilamellar and multilamellar liposomes of the composition according to the present invention.

Complexing of substances includes, but not limited to, non-covalent binding, e.g. electrostatic interaction, hydrophobic attraction of macromolecules to liposomes of same or opposite charge.

Macromolecules may be used as a further adjuvant. A macromolecule is defined as a very large molecule, such as a protein, consisting of many smaller structural units linked together; also molecules such as DNA and RNA which are very large and complex are defined as macromolecules. The term is applied to the four conventional biopolymers (nucleic acids, proteins, carbohydrates, and lipids), as well as non-polymeric molecules with large molecular mass. The use of macromolecules in the present invention is including but not limited to oligonucleotides, peptides, proteins, carbohydrates and lipids.

The present invention discloses the homogenous mixing of liposomal lipid components, such as cationic surfactants like DDA and glycolipids like MMG, before hydration and after hydration the potential for subsequent complexing with other macromolecules. Mixing of the lipid components with other macromolecules (e.g. oligonucleotides) can also be done before hydration and have the high shear-mixing do the complexing. Complexes between cationic lipids and in particular negatively charged macromolecules (e.g. oligonucleotides) are generally thermodynamically unstable resulting in the formation of larger aggregates over time, a broad size distribution and structural heterogeneity. The binding of highly charged polyelectrolytes to oppositely charged liposomes promotes membrane destabilization per se. Charge neutralization and concomitant reduction of the outer surface area leads to a gradient in lateral pressure in the direction of the bilayer normal. This generates a bending moment, which in turn contributes to the destabilisation of the liposomes.

It should be noted that embodiments and features described in the context of one of the aspects of the present invention also apply to the other aspects of the invention.

One aspect of the present invention relates to a Liposomal composition comprising the cationic lipid dimethyldioctadecylammonium (DDA) and at least one immunomodulator for use as a medicament in a subject.

Another aspect of the present invention relates to a Liposomal composition comprising the cationic lipid dimethyldioctadecylammonium (DDA) and at least one immunomodulator for use in the prevention or treatment of pathogenic infection of the respiratory tract in a subject.

One embodiment of the present invention relates to a liposomal composition for use in the prevention of a pathogenic infection, wherein the pathogenic infection is selected from viral infection or bacterial infection.

The pathogenic infection may be an infection in the respiratory tract, such as the upper or lower respiratory tract.

Thus, in further embodiment of the present invention, the pathogenic infection may be a virus infection of the respiratory tract caused by a virus selected from the group consisting of a picornavirus, rhinovirus, coronavirus, such as MERS-corona virus, SARS-coronavirus, such as SARS-CoV-2, influenza virus, human parainfluenza virus, human respiratory syncytial virus, adenovirus, enterovirus, and metapneumovirus.

In another embodiment of the present invention, the pathogenic infection is a bacterial infection of the respiratory tract caused by a bacteria selected from the group selected from Chlamydia pneumoniae, Streptococcus pneumoniae, Streptococcus pyrogenes, Haemophilus influenza, Moraxella catarrhalis and a mycobacterium, such as M. tuberculosis, M. bovis, M. africanum, M. canetti, and M. microti. Burkholderia Sp.

One embodiment of the present invention relates to the liposomal composition for use as disclosed herein, which further comprises at least one glycolipid, such as monomycoloyl glycerol analogue (MMG).

The immunomodulator may be selected from immunomodulators that can signal to inhibit viral replication or immunomodulators that can signal to activate or recruit proinflammatory cells that eliminate pathogens, such as granulocytes, macrophages and NK cells.

The immunomodulator may be selected from the group consisting of STING agonists such as cyclic-GMP-AMP (cGAMP), cAMP, c-di-AMP, cGMP and c-di-GMP, TLR2 agonists, such as zymosan, TLR3 agonists such as double-stranded ribonucleic acids (dsRNAs) like polyinosinic acid:polycytidylic acid (poly(I:C)), TLR4 agonists, such as MPL-A, TLR5 agonists such as flagellin, TLR7/8 agonists, such as resiquimod, imiquimod, gardiquimod and also including lipidated analogs, C-type lectin receptors, such as cord factor (TDM) or the synthetic analogue TDB, nucleotide-binding oligomerization domain (NOD) receptor agonists such as MDP. Preferably, the immunomodulator is polyinosinic acid:polycytidylic acid (poly(I:C)), more preferred a synthetic double-stranded Poly (I:C) RNA analogue.

In an embodiment of the present invention, the immunomodulator is preferably a STING agonist selected from the group consisting of cGAMP, cAMP, cGMP, c-di-AMP and c-di-GMP, more preferably c-di-GMP.

In another embodiment of the present invention, the monomycoloyl glycerol analogue (MMG) of the liposomal composition is preferably the analogue 3-hydroxy-2-tetradecyl-octadecanoic acid-2,3-dihydroxypropyl ester.

In a further embodiment of the present invention, the DDA of liposomal composition is preferably dimethyldioctadecylammonium bromide.

Another embodiment of the present invention relates to the liposomal composition as disclosed herein comprising the cationic lipid dimethyldioctadecyl-ammonium (DDA) and monomycoloyl glycerol (MMG).A preferred embodiment of the present invention relates to the liposomal composition as disclosed herein comprising 1-3 mg/ml DDA, and 0.1-1.0 mg/ml MMG-1. The liposomal composition preferably comprises 2.5 mg/ml DDA and 0.5 mg/ml MMG-1.

A further embodiment of the present invention relates to the liposomal composition comprising 0.1-1.0 mg/ml Poly (I:C), preferably 0.5 mg/ml poly (I:C) (corresponding to the content of CAF®09), especially preferred 0.125 mg/ml poly (I:C) (corresponding to the content of CAF®09b).

Another embodiment of the present invention relates to the liposomal composition as disclosed herein, wherein the liposomal composition comprises the cationic lipid dimethyldioctadecyl-ammonium (DDA), monomycoloyl glycerol (MMG) and at least one further immunomodulator.

One embodiment of the present invention relates to the liposomal composition as disclosed herein, wherein the liposomal composition comprises the cationic lipid dimethyldioctadecyl-ammonium (DDA), monomycoloyl glycerol (MMG), poly (I:C) and at least one further immunomodulator.

A further embodiment of the present invention relates to the liposomal composition as disclosed herein, wherein the liposomal composition comprises the immunomodulatory c-di-GMP.

Another embodiment of the present invention relates to the liposomal composition as disclosed herein, wherein the liposomal composition comprises the cationic lipid dimethyldioctadecyl-ammonium (DDA), monomycoloyl glycerol (MMG) and c-di-GMP.

A further embodiment of the present invention relates to the liposomal composition as disclosed herein, wherein the liposomal composition comprises comprises the cationic lipid dimethyldioctadecyl-ammonium (DDA), monomycoloyl glycerol

(MMG), poly (I:C) and c-di-GMP.

In another embodiment, the liposomal composition further comprises a STING agonist, preferably c-di-GMP, in a concentration of 0.025-2.5 g/ml, such as in a concentration of 0.05-2.0 mg/ml, such as in a concentration of 0.05-1.5 mg/ml, such as in a concentration of 0.05-1.0 mg/ml, such as in a concentration of 0.025-0.5 mg/ml, preferably in a concentration of 0.05-0.2 mg/ml, such as 0.1-0.2 mg/ml.

In a further embodiment, the liposomal composition comprises 2.5 mg/ml DDA, 0.5 mg/ml MMG-1 and 0.1 mg/ml c-di-GMP.

In another embodiment of the present invention, the liposomal composition as disclosed herein is administered by systemic administration, nasal administration and/or pulmonary administration. The liposomal composition may also be administered by both systemic administration and nasal administration.

In a further embodiment, the liposomal composition may be administered by nasal and/or pulmonary administration after exposure to a pathogen to treat the early infection of the respiratory tract.

In yet another embodiment of the present invention, the liposomal composition is administered by nasal or pulmonary administration to prevent infection of the respiratory tract. The liposomal composition may also be administered by both nasal and pulmonary administration. In a further embodiment, the liposomal composition is administered one to seven times per week, such as two, three, four, five or six times per week.

Accordingly, the subject, who may benefit especially from the present invention, may have chronic obstructive pulmonary disease (COPD), asthma cystic fibrosis, or another condition that results in compromised respiratory function compared to a healthy subject. Subjects with lung cancer may also receive the liposomal composition according to the present invention. In certain embodiments, the subjects may be smokers, with either or both of a past history of smoking or an ongoing use of cigarettes or other smoking products. These subjects are vulnerable to upper respiratory infections, so administration of a disclosed composition could potentially prevent upcoming common cold symptoms or illnesses and thus prevent exacerbations of their underlying illnesses and symptoms.

In one embodiment of the present invention, the subject is selected from the group consisting of humans of all ages, other primates (e.g., cynomolgus monkeys, rhesus monkeys); mammals in general, including commercially relevant mammals, such as cattle, pigs, horses, sheep, goats, cats and dogs, as well as birds. Preferably, the subject is a human.

In another embodiment of the present invention, the subject is an individual with compromised/reduced immunity in the airways.

In a further embodiment of the present invention, the subject has a disease selected from chronic obstructive pulmonary disease, asthma, cystic fibrosis, lung cancer and diabetes. Moreover, the subject may have a past history of smoking or may be a current smoker.

In another embodiment, the subject is a healthy subject of the population or a healthy subject, who are exposed to pathogenes and in need of protection against infection, such as health personal.

Yet another aspect of the present invention relates to a device for nasal or pulmonary administration comprising the liposomal composition.

Especially preferred liposomal composition according to the present invention are CAF®04, CAF®09 and CAF®09b.

CAF®04 is a two-component cationic liposomal adjuvant system developed by Statens Serum Institut, Denmark, and composed of cationic DDA liposomes with the glycolipid and immunomodulator, monomycoloyl glycerol (MMG), incorporated into the bilayer of the liposomal membrane.

CAF®09 and CAF®09b are a three-component cationic liposomal adjuvant system developed by Statens Serum Institut, Denmark, and composed of cationic DDA liposomes with the glycolipid and immunomodulator, monomycoloyl glycerol (MMG), incorporated into the bilayer of the liposomal membrane and the immunomodulator, poly I:C, bound to the liposome surface.

Content of CAF®04, CAF®09 and CAF®09b:
CAF®04: 2500/500 μg/mL DDA:MMG-1 dispersed in 10 mM Tris+4% glycerol adjusted to pH 7.0.
CAF®09: 2500/500/500 μg/mL DDA:MMG-1:poly (I:C) dispersed in 10 mM Tris+4% glycerol adjusted to pH 7.0.
CAF®09b: 2500/500/125 ug/mL DDA:MMG-1:poly (I:C) dispersed in 10mM Tris+4% glycerol adjusted to pH 7.0.

All patent and non-patent references cited in the present application, are hereby incorporated by reference in their entirety.

The invention will now be described in further details in the following non-limiting examples.

### EXAMPLES

The examples are meant to illustrate the invention.

CAF®04 and CAF®09b are used as an exemplary liposomal composition according to the invention in the below examples.

CAF®04 is a two-component cationic liposomal adjuvant system developed by Statens Serum Institut, Denmark, and composed of cationic DDA liposomes with the glycolipid and immunomodulator, monomycoloyl glycerol (MMG), incorporated into the bilayer of the liposomal membrane.

The strength of CAF®04 2500/500 is 2500 μg DDA per mL and 500 μg MMG per mL dispersed in 10mM Tris+4% glycerol adjusted to pH 7.0.

CAF®09b is a three-component cationic liposomal adjuvant system developed by Statens Serum Institut, Denmark, and composed of cationic DDA liposomes with the glycolipid and immunomodulator, monomycoloyl glycerol (MMG), incorporated into the bilayer of the liposomal membrane and the immunomodulator, poly I:C, bound to the liposome surface.

The strength of CAF®09b 2500/500/125 is 2500 μg DDA per mL, 500 μg MMG per mL, and 125 μg Poly I:C per mL dispersed in 10 mM Tris+4% glycerol adjusted to pH 7.0.

### Example 1

### Influx of Innate Immune Cells

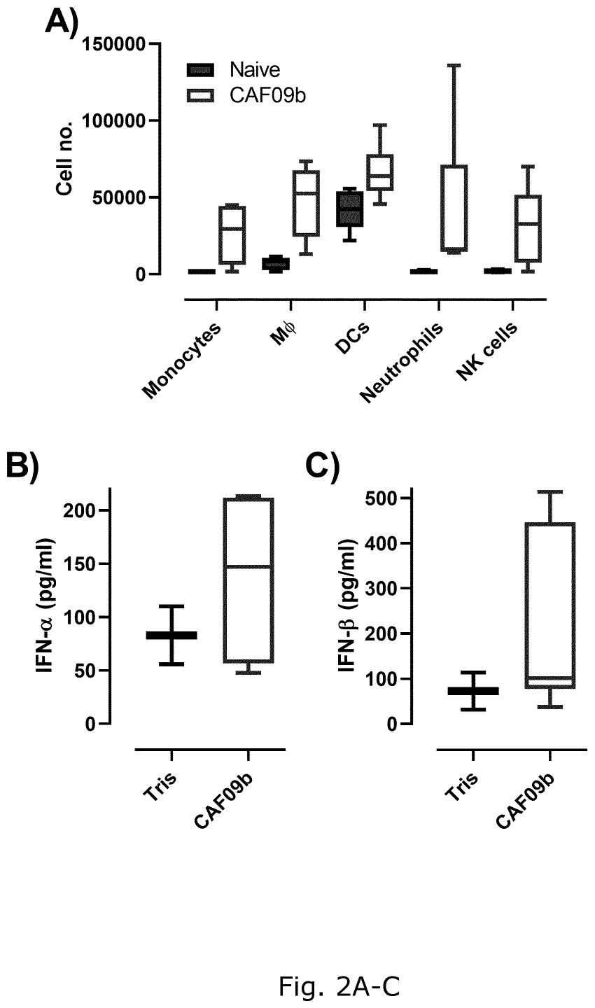

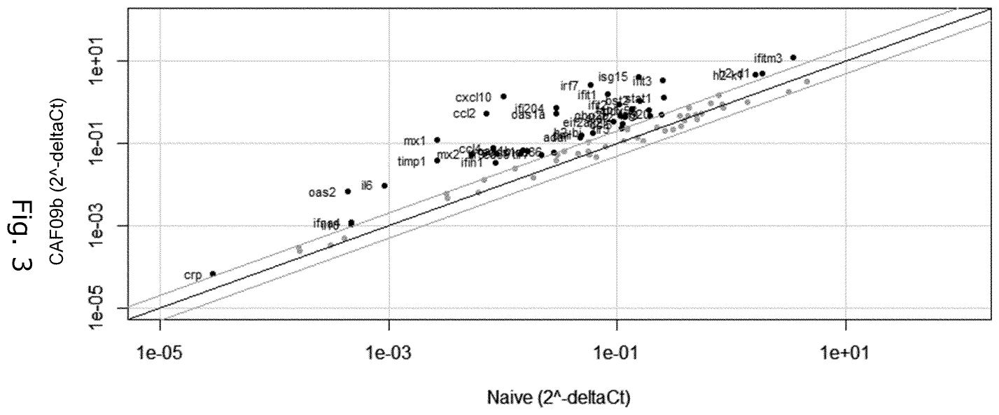

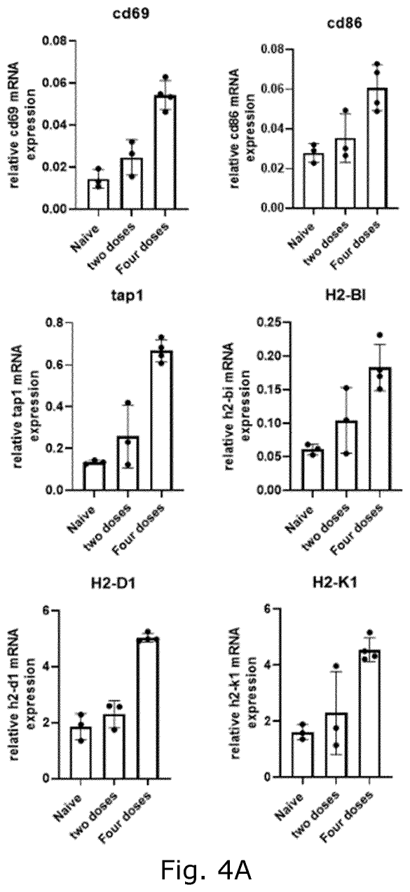

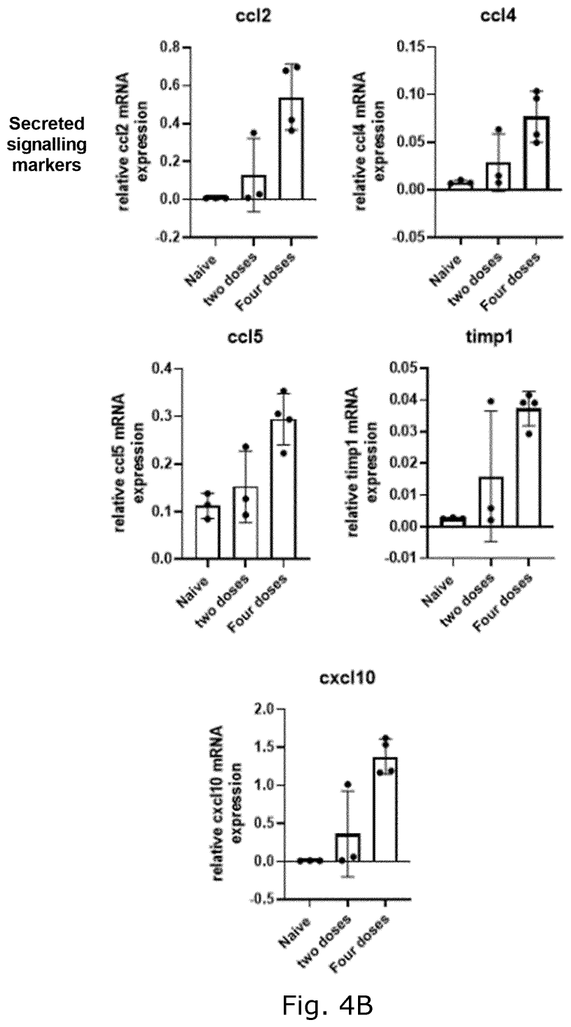

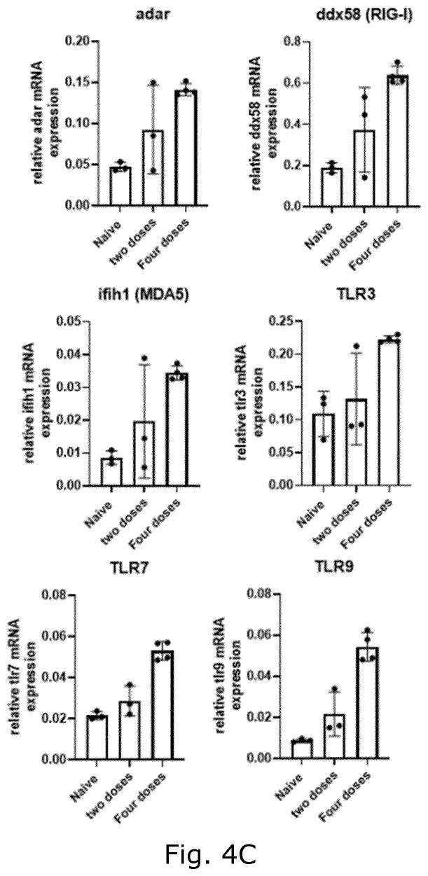

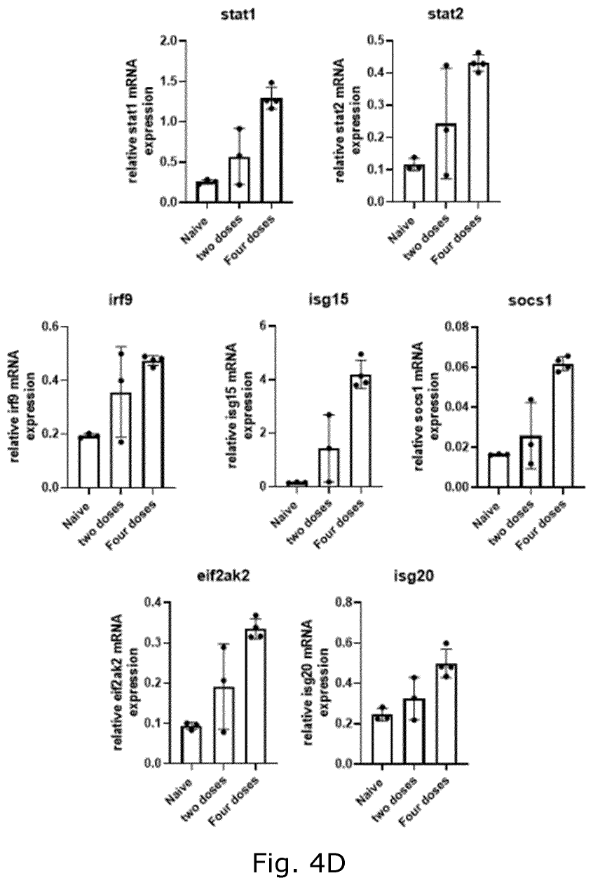

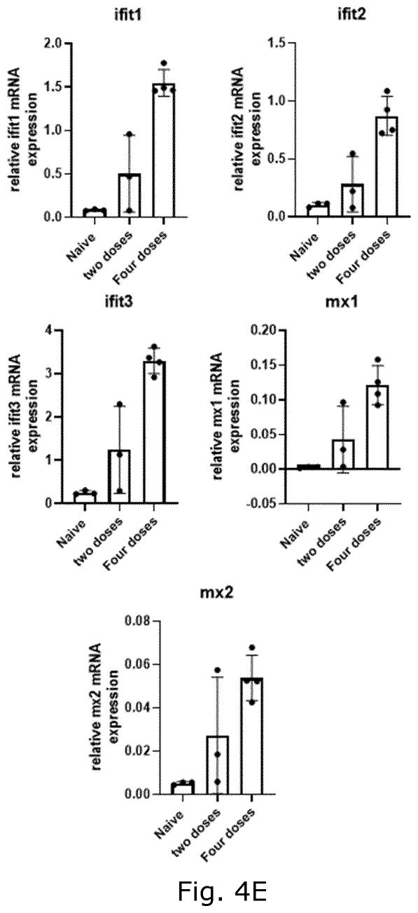

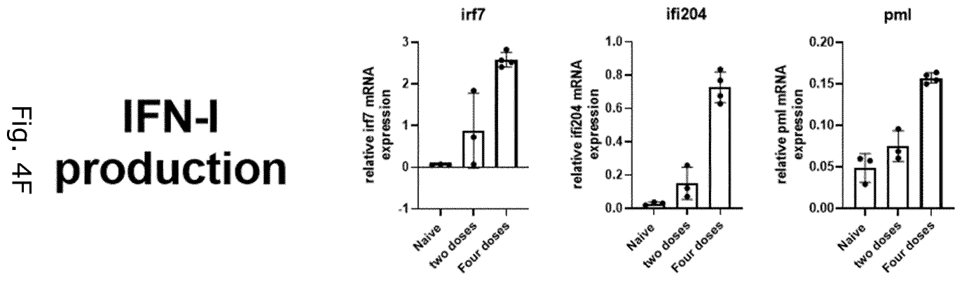

CB6F1 mice were treated intranasally with CAF®09b twice with 72 hours interval. 24 hours later the study was terminated and perfused lungs were isolated, and flow cytometry analysis was conducted to determine the influx of innate immune cells (FIG. 2A). Intranasal administration of CAF®09b facilitated an increased influx of monocytes (MHC II+, CD11b+, Ly6C+), Mφ) (MHC II+, F4/80+), DCs (MHC II+, CD11c+), Neutrophils (Ly6G+) and NK (NK1.1+) cells into the lungs. Electrochemo-luminiscence (MSD) analysis were conducted to measure IFN-I in the lung cell supernatant. Both IFN-α (FIG. 2B) and IFN-β (FIG. 2C) were elevated after CAF®09b treatment.

### Example 2

### Proinflammatory and Type 1-Interferon Markers

CB6F1 mice were treated twice i.n. with CAF®09b with 72h interval or four times with 24h interval. 24h later the study was terminated and perfused lungs were isolated, and QPCR analysis using Qiagen RT2 Profiler PCR Array for proinflammatory and type 1-interferon markers was conducted to determine genes upregulated after treatment. The data show upregulation of genes related to both type 1-interferon and proinflammatory responses including surface activation markers: CD69, CD86, Tap1, H2-BI, H2-DI and H2-KI; Secreted signalling markers: CCL2, CCL4, CCLS, Timp1 and CXCL10; IFN-I related pattern recognition receptors: Adar, RIG-I, MDA-5, TLR3, TLR7 and TLR9; IFN-I regulated genes: stat1, stat2, irf9, isg15, socs1, eif2ak2 and isg20; Inhibition of viral translation and replication: ifit1, ifit2, ifit3, mx1 and mx2; IFN-I production: irf7, ifi204 and pmI (FIG. 3+4A-F). These data show that CAF®09b induces the innate immune response required for protection against viral protection.

### Example 3

### Weight and Survival Following Influenza Infection

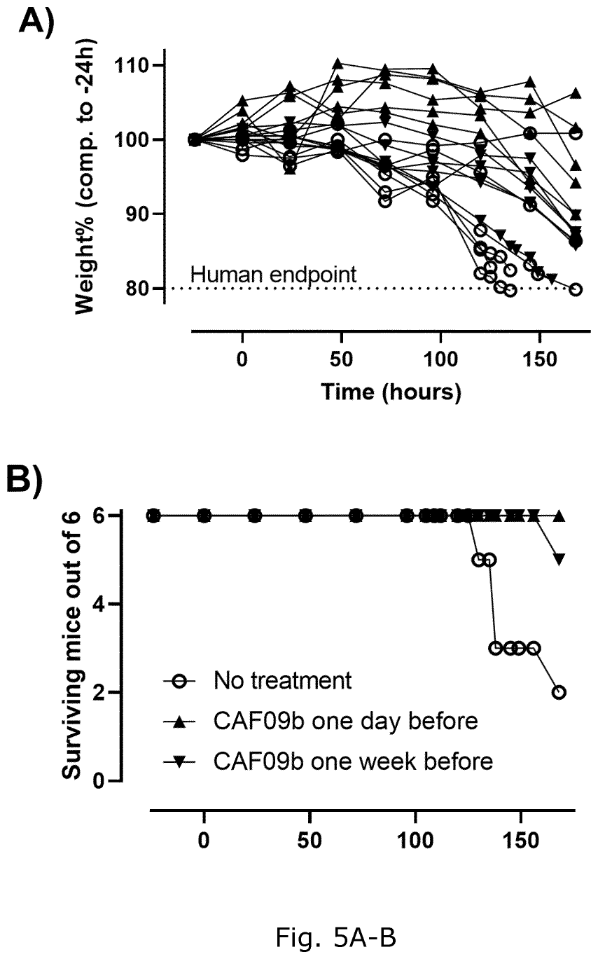

CB6F1 mice were treated twice i.n. with CAF®09b with 3 days interval. Mice received an influenza infection 4 and 10 days after the first immunization. Weight (FIG. 5A) and survival (FIG. 5B) were monitored for 7 days. 4/6 mice in the naive group but only 1/6 mice in the CAF®09b group treated one week before reached the human endpoint weight and had to be taken down. None of the mice treated with CAF®09b one day before challenge reached the human endpoint during the study. This study suggests that treatment efficacy last for at least a week.

### Example 4

### HA Specific Antibodies

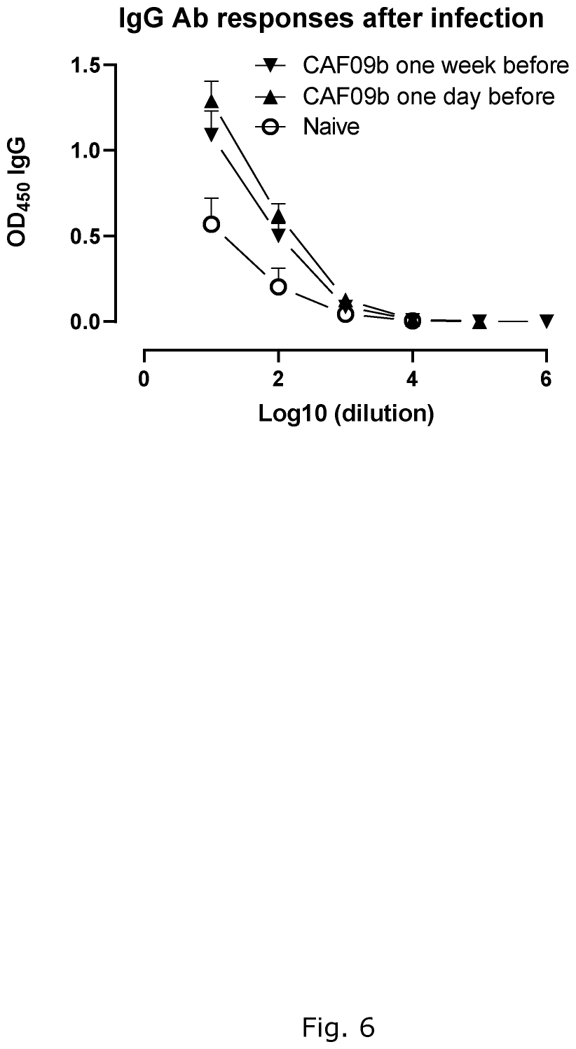

Blood was withdrawn from mice in example 3 at the day they were terminated to investigate whether innate protection resulted in reduced adaptive immunity. Influenza HA specific antibodies were measured using ELISA (FIG. 6).

The data shows that CAF®09b treatment despite reduced symptoms does not affect the infection driven induction of acquired immunity, as the antibody levels after infection in the CAF®09b treated groups were at level with the non-treated group—if not higher.

### Example 5

### CAF®04 in Combination with STING Agonist

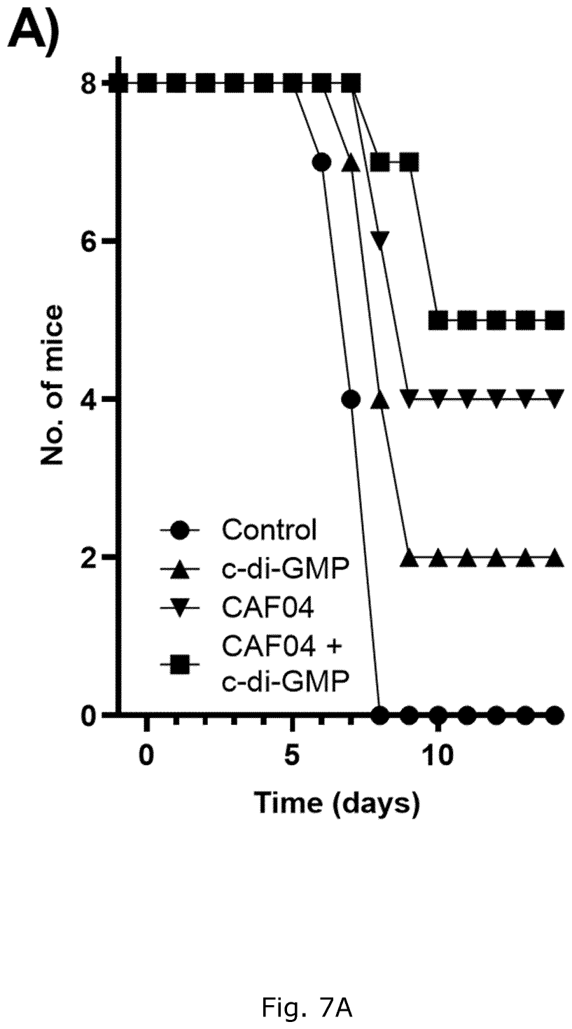

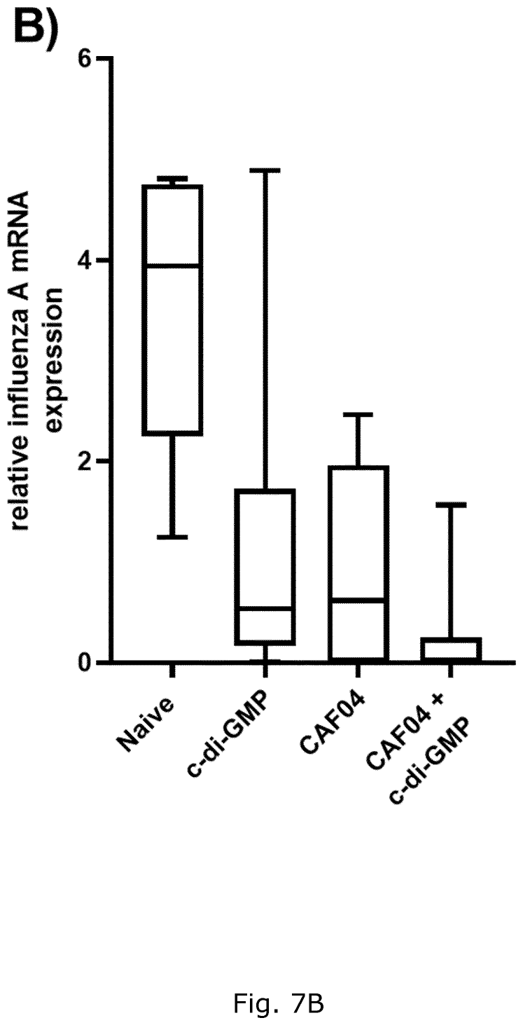

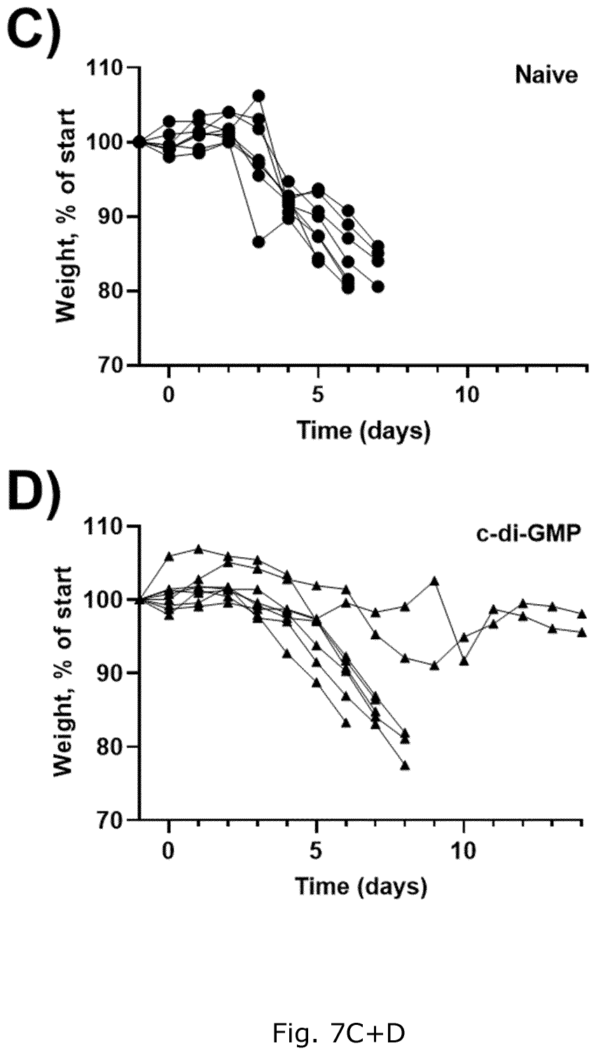

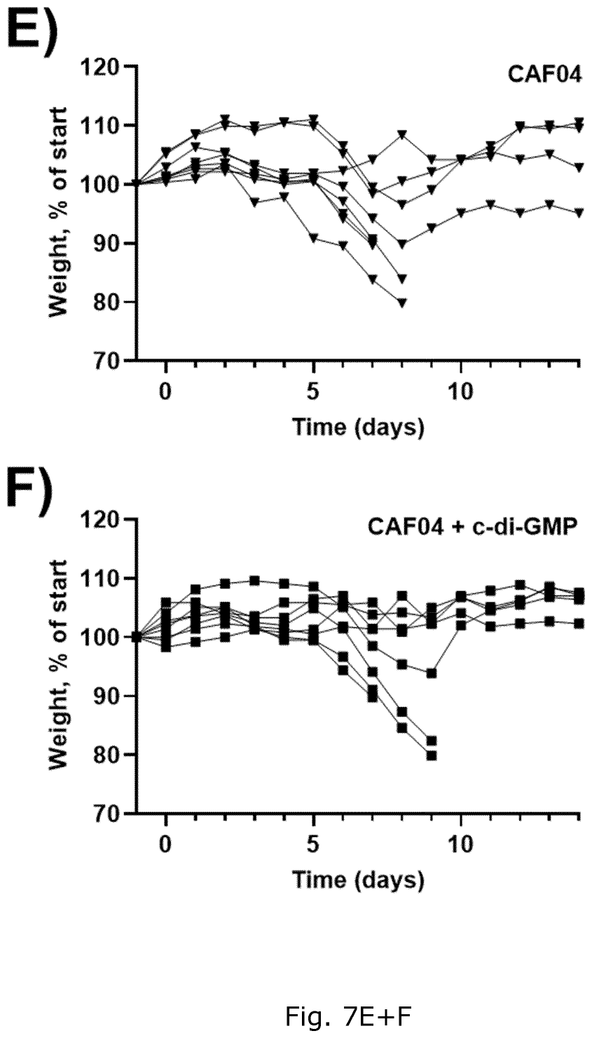

Validation of CAF®04 (2500/500 μg/mL DDA/MMG-1) was performed in combination with the STING (Stimulator of Interferon Genes) agonist c-di-GMP in a concentration of 100 μg/ml, which can control the transcription of host defense genes, including pro-inflammatory cytokines and chemokines, and type I interferons (IFNs). Mice were treated four times with 24-hours interval with either c-di-GMP alone, CAF®04 alone or CAF®04+c-di-GMP and received an influenza infection 5 days after the first immunization. Survival (FIG. 7A), viral load (FIG. 7B) and weight (FIG. 7C-F) were monitored for 14 days. All mice in the naive group (FIG. 7A,C), 6/8 mice in the c-di-GMP group (FIG. 7A,D) and 4/8 mice in the CAF®04 group (FIG. 7A,E), but only 3/8 mice in the CAF®04+c-di-GMP group reached the human endpoint weight and had to be taken down. This study suggests that CAF®04 and c-di-GMP synergize in treatment efficacy, resulting in superior efficacy by combination. This is also reflected in the ability to reduce viral load (FIG. 7B).

### Example 6

### CAF®09b in Combination with STING Agonist

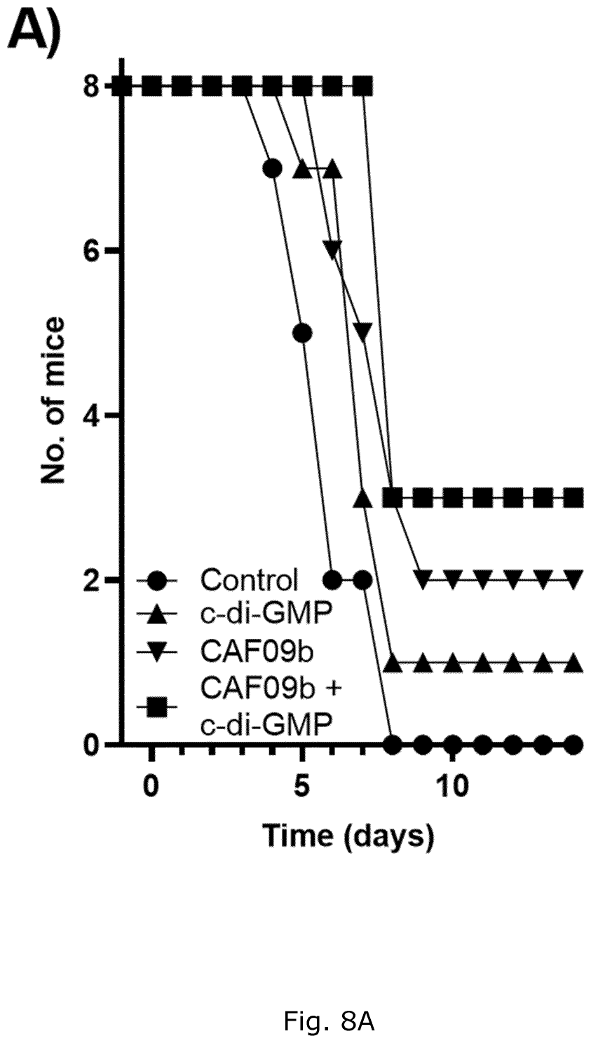

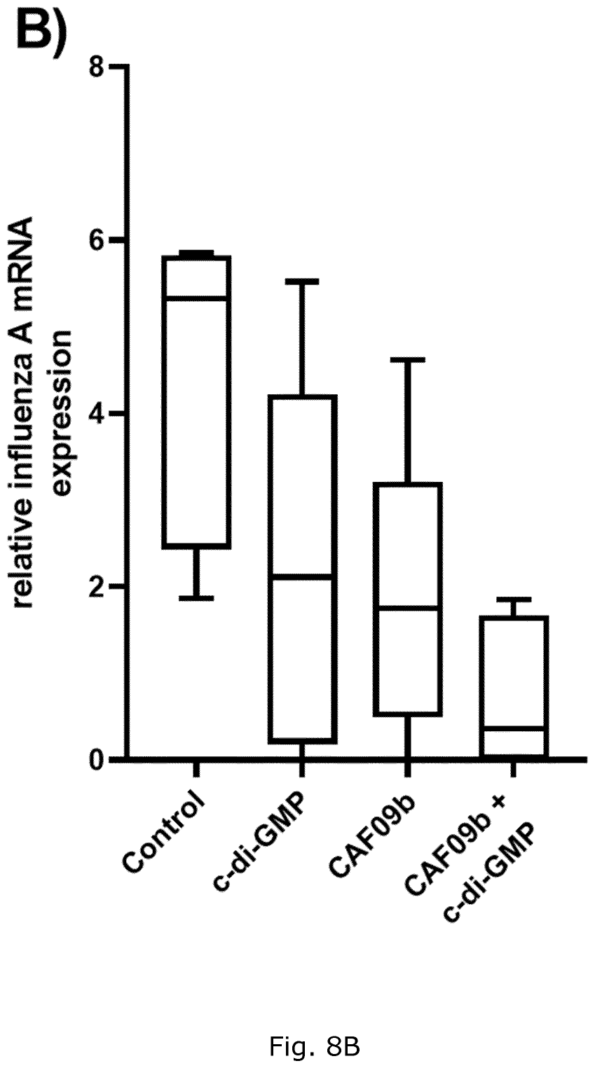

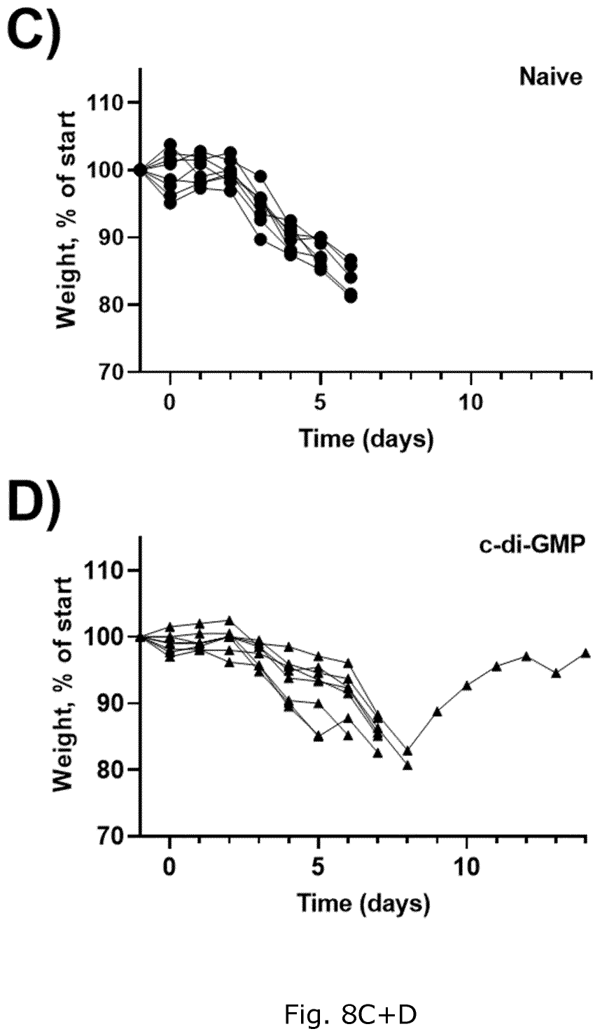

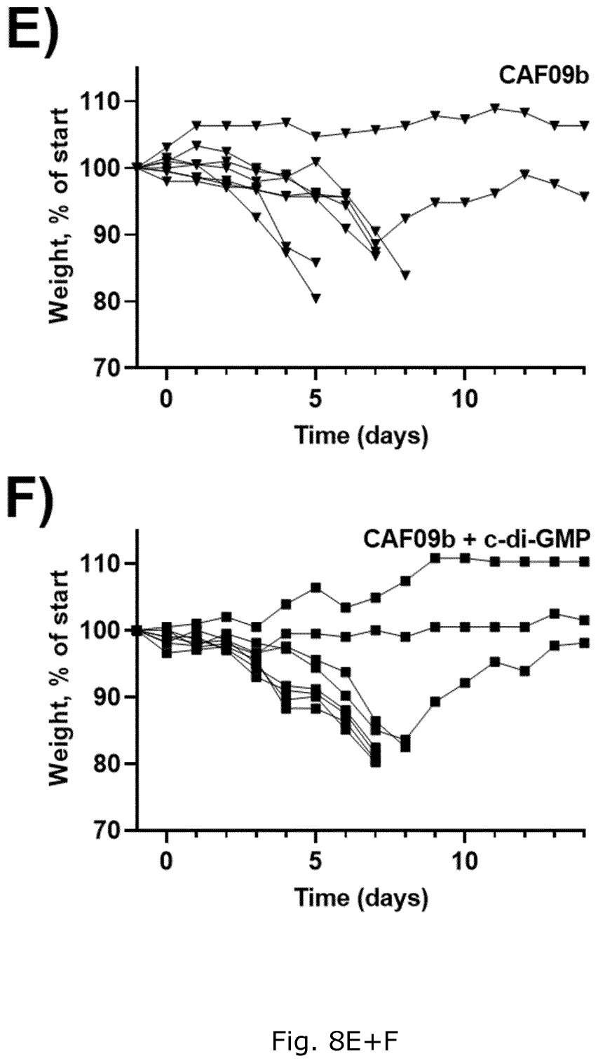

Validation of CAF®09b (2500/500/125 μg/mL DDA/MMG-1/polyIC) was performed in combination with the STING (Stimulator of Interferon Genes) agonist c-di-GMP in a concentration of 100 μg/ml, which can control the transcription of host defense genes, including pro-inflammatory cytokines and chemokines, and type I interferons (IFNs). Mice were treated two times with 3-days interval with either c-di-GMP alone, CAF®09b alone or CAF®09b +c-di-GMP and received a high dose influenza infection 5 days after the first immunization. Survival (FIG. 8A), Viral load (FIG. 8B) and Weight (FIG. 8C-F) were monitored for 14 days. All mice in the naive group (FIG. 8A,C), 7/8 mice in the c-di-GMP group (FIG. 8A,D) and 6/8 mice in the CAF®09b group (FIG. 8A,E), but only 5/8 mice in the CAF®09b+c-di-GMP group reached the human endpoint weight and had to be taken down. This study suggests that CAF®09b and c-di-GMP synergize in treatment efficacy, resulting in superior efficacy by combination. This is also reflected in the ability to reduce viral load (FIG. 8B).

### Example 7

### Combination of CAF®04, CAF®09 and STING Agonist

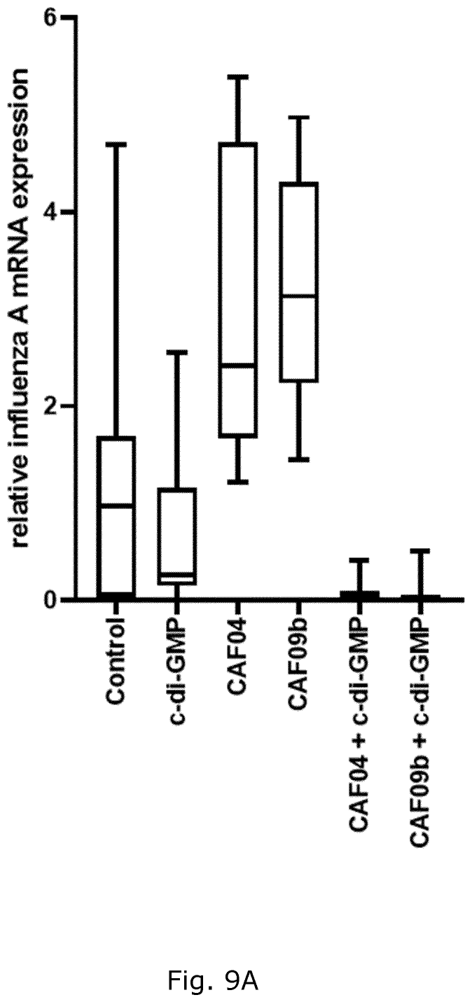

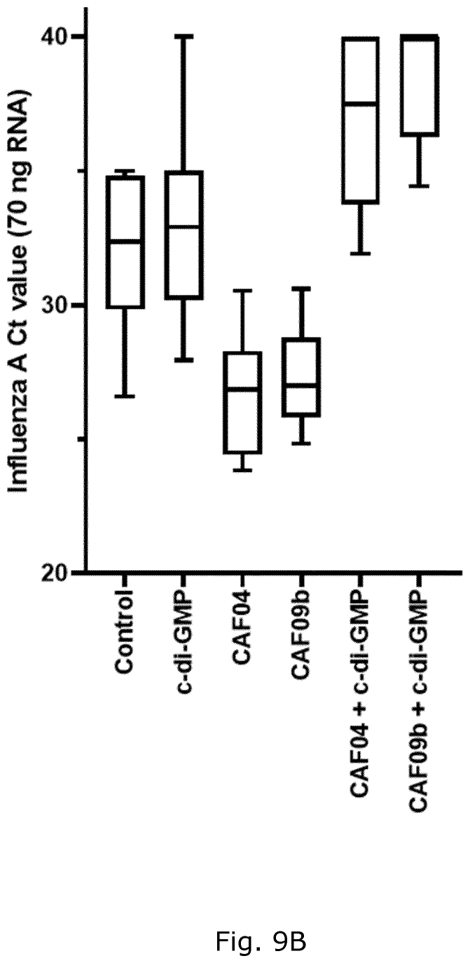

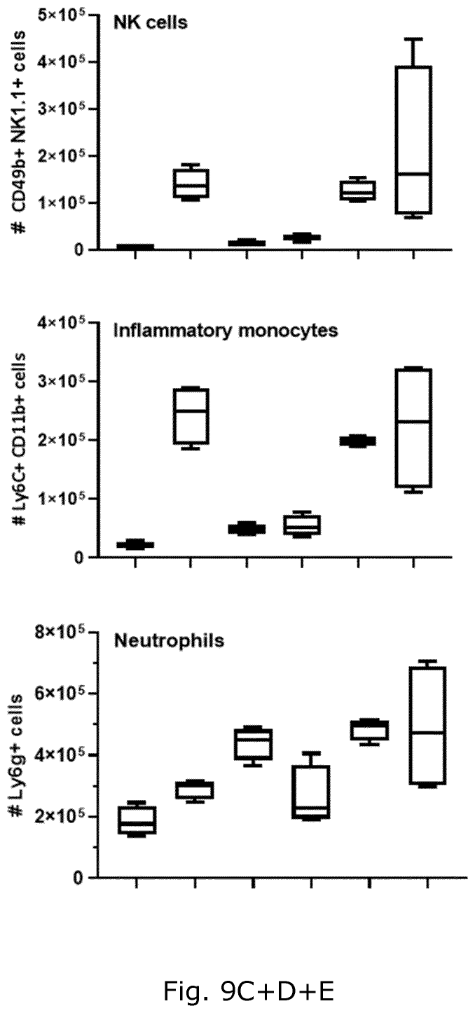

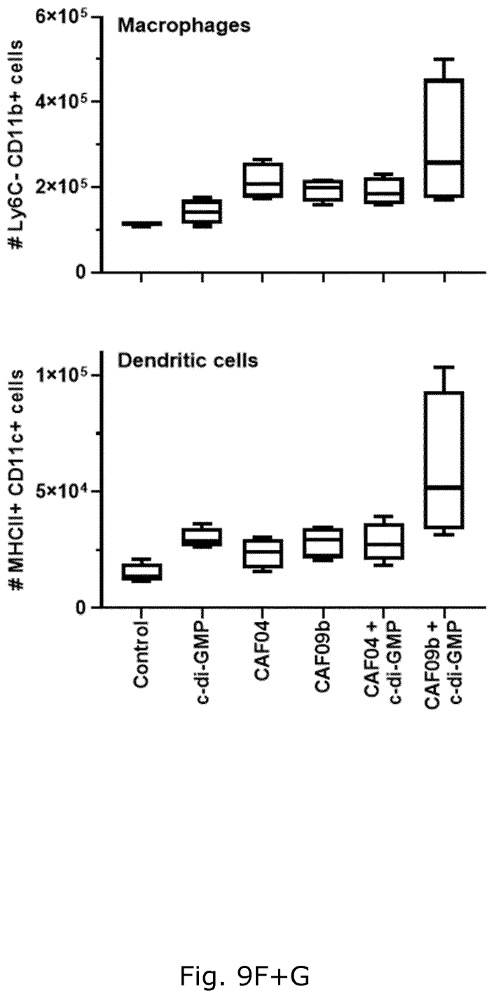

Validation of CAF®04 (2500/500 μg/mL DDA/MMG-1) and CAF®09b (2500/500/125 μg/mL DDA/MMG-1/polylC) was performed in combination with the STING agonist c-di-GMP in a concentration of 100 μg/ml, for the ability to block viral replication in the nose. Mice were treated two times with 3-days interval with either c-di-GMP, CAF®04, CAF®09b, CAF®04+c-di-GMP, CAF®09b+c-di-GMP and received a high dose influenza infection 5 days after the first immunization. Viral load, measured by relative Influenza mRNA expression (FIG. 9A) and CT values (FIG. 9B; CT value equals the total number of cycles required to find RNA, and each positive test has its own CT value; If no RNA is found within 37 to 40 cycles, the test is negative), were analysed in the nose two days after challenge. This study suggests that c-di-GMP facilitate reduction of viral replication in the nose, which is further promoted by combination with CAF®04 and CAF®09b and that the combination thus synergizes in treatment efficacy by preventing virus proliferation in the nose. This correlates with the ability of c-di-GMP containing adjuvants to recruit increased levels of activated NK cells (FIG. 9C), inflammatory monocytes (FIG. 9D) and neutrophils (FIG. 9E) before infection. Only the CAF®09b+c-di-GMP increased the levels of macrophages (FIG. 9F) and dendritic cells (FIG. 9G) significantly.

### Example 10

### CAF®09b and SARS-CoV-2

Syrian Gold Hamsters were treated twice i.n. with CAF®09b with 3 days interval.

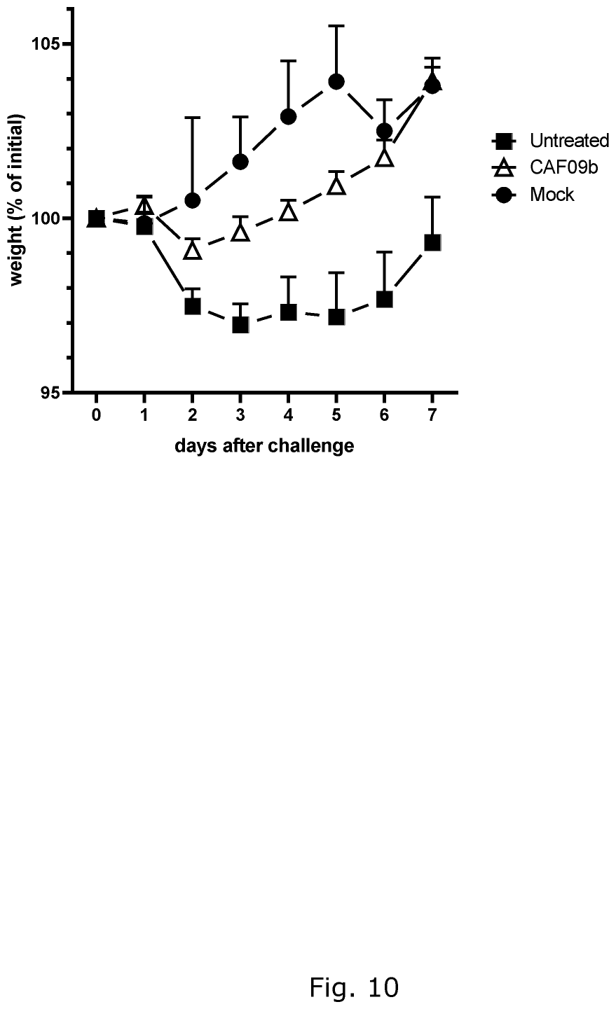

The Hamsters received a SARS-CoV-2 infection (1.9×10⁵TCID₅₀) 5 days after the first immunization. Weight were monitored for 7 days as an indication of health status. Those hamsters not receiving CAF®09b treatment experienced a significantly larger weight loss than the CAF®09b treated group and had not regained the weight at day 7, which was the case for the CAF®09b treated group (FIG. 10).

### REFERENCES

1 Hackett, C. J. Innate immune activation as a broadspectrum biodefense strategy: Prospects and research challenges. J ALLERGY CLIN IMMUNOL 112, 8 (2003).

2 Hogan, R. J. et al. Resolution of primary severe acute respiratory syndrome-associated coronavirus infection requires Stat1. J Virol 78, 11416-11421, doi:10.1128/JVI.78.20.11416-11421.2004 (2004).

3 Taylor, D. R. Obstacles and advances in SARS vaccine development. Vaccine 24, 863-871, doi:10.1016/j.vaccine.2005.08.102 (2006).

4 Uematsu, S. & Akira, S. Toll-like receptors and Type I interferons. J Biol Chem 282, 15319-15323, doi:10.1074/jbc.R700009200 (2007).

5 Lau, Y. F., Tang, L. H., McCall, A. W., Ooi, E. E. & Subbarao, K. An adjuvant for the induction of potent, protective humoral responses to an H5N1 influenza virus vaccine with antigen-sparing effect in mice. J Virol 84, 8639-8649, doi:10.1128/JVI.00596-10 (2010).

6 Lau, Y. F., Tang, L. H., Ooi, E. E. & Subbarao, K. Activation of the innate immune system provides broad-spectrum protection against influenza A viruses with pandemic potential in mice. Virology 406, 80-87, doi:10.1016/j.viro1.2010.07.008 (2010).

7 Martins, K. A., Bavari, S. & Salazar, A. M. Vaccine adjuvant uses of poly-IC and derivatives. Expert Rev Vaccines 14, 447-459, doi:10.1586/14760584.2015.966085 (2015).

8 Pedersen, G. K., Andersen, P. & Christensen, D. Immunocorrelates of CAF family adjuvants. Semin Immunol 39, 4-13, doi:10.1016/j.smim.2018.10.003 (2018).

9 Abraham, S. et al. Safety and immunogenicity of the chlamydia vaccine candidate CTH522 adjuvanted with CAF01 liposomes or aluminium hydroxide: a first-in-human, randomised, double-blind, placebo-controlled, phase 1 trial. Lancet Infect Dis 19, 1091-1100, doi:10.1016/51473-3099(19)30279-8 (2019).

10 Vrieze, J. d. Can a century-old TB vaccine steel the immune system against the new coronavirus? Science (2020).

11. Virology. 2009 Dec. 20; 395(2): 210-222.

1. (canceled)

2. A method of treating pathogenic infection of the respiratory tract in a subject comprising administering a Liposomal composition comprising the cationic lipid dimethyldioctadecyl-ammonium (DDA) and at least one immunomodulator to the subject in need thereof.

3. The method according to claim 2, wherein the liposomal composition comprises the cationic lipid dimethyldioctadecyl-ammonium (DDA) and monomycoloyl glycerol (MMG).

4. The method according to claim 3, wherein the liposomal composition comprises 1-3 mg/ml DDA and 0.1-1.0 mg/ml MMG, or wherein the liposomal composition comprises 2.5 mg/ml DDA and 0.5 mg/ml MMG.

5. (canceled)

6. The method according to claim 2, wherein the liposomal composition comprises the cationic lipid dimethyldioctadecyl-ammonium (DDA), monomycoloyl glycerol (MMG) and at least one further immunomodulator wherein the immunomodulator is selected from a polyinosinic acid:polycytidylic acid (poly(LC)), and a synthetic double-stranded Poly (I:C) RNA analogue.

7. (canceled)

8. The method according to claim 6, comprising 0.05-1.0 mg/ml Poly (I:C), or 0.1-0.5 mg/ml poly (I:C), or 0.5 mg/ml poly (I:C) or 0.125 mg/ml poly (I:C).

9. (canceled)

10. The method according to claim 2, wherein the liposomal composition comprises poly (I:C) and at least one further immunomodulator.

11. The method according to claim 10, wherein the immunomodulator is selected from the group consisting of one or more of a STING agonists, cGAMP, cAMP, c-di-AMP, cGMP and c-di-GMP, a TLR2 agonist, zymosan, a TLR3 agonist, a double-stranded ribonucleic acids (dsRNAs), polyinosinic acid:polycytidylic acid (poly(I:C)), a TLR4 agonist, MPL-A, a TLR5 agonist, flagellin, a TLR7/8 agonist, resiquimod, imiquimod, gardiquimod, a lipidated analog, a C-type lectin receptors, cord factor (TDM), a synthetic analogue TDB, a nucleotide-binding oligomerization domain (NOD) receptor agonist, and MDP.

12. The method according to claim 11, wherein the liposomal composition comprises immunomodulator c-di-GMP, in a concentration of 0.05-0.2 mg/ml, or 0.1 mg/ml, or
wherein the liposomal composition comprises the cationic lipid dimethyldioctadecyl-ammonium (DDA), monomycoloyl glycerol (MMG) and 0.5-0.2mg/ml c-di-GMP, or
wherein the liposomal composition comprises the cationic lipid dimethyldioctadecyl-ammonium (DDA), monomycoloyl glycerol (MMG) and 0.1 mg/ml c-di-GMP, or
wherein the liposomal composition comprises comprises the cationic lipid dimethyldioctadecyl-ammonium (DDA), monomycoloyl glycerol (MMG), and poly (I:C).

13. (canceled)

14. (canceled)

15. The method according to claim 2, wherein the pathogenic infection is selected from a respiratory tract viral infection and, or, bacterial infection, said infection being present in the upper respiratory tract or lower respiratory tract or both.

16. (canceled)

17. The method according to claim 2, wherein the pathogenic infection is a virus infection of the respiratory tract caused by a virus selected from the group consisting of a picornavirus, rhinovirus, coronavirus, MERS-corona virus, SARS-coronavirus, SARS-CoV-2, influenza virus, human parainfluenza virus, human respiratory syncytial virus, adenovirus, enterovirus, and metapneumovirus.

18. The method according to claim 2, wherein the pathogenic infection is a bacterial infection of the respiratory tract caused by a bacteria selected from the group selected from Chlamydia pneumoniae, Streptococcus pneumoniae, Streptococcus pyogenes, Haemophilus influenza, Moraxella catarrhalis and a mycobacterium, such as M. tuberculosis, M. bovis, M. africanum, M. canetti, and M. microti. Burkholderia Sp.

19. The method according to claim 2, wherein the immunomodulator is selected from immunomodulators that can signal to inhibit viral replication or immunomodulators that can signal to activate or recruit proinflammatory cells that eliminate pathogens, such as granulocytes, macrophages and NK cells.

20. The method according to claim 2, wherein the monomycoloyl glycerol analogue (MMG) is the analogue 3-hydroxy-2-tetradecyl-octadecanoic acid-2,3-dihydroxypropyl ester.

21. The method according to claim 2, wherein the DDA is dimethyldioctadecylammonium bromide.

22. The method according to claim 2, which do wherein said composition does not comprise an antigen.

23. (canceled)

24. The method according to claim 2, wherein the liposomal composition is administered by systemic administration, nasal administration and/or pulmonary administration or.
wherein the liposomal composition is administered by both systemic administration and nasal administration.

25. (canceled)

26. The method according to claim 2, wherein the liposomal composition is administered by nasal and/or, pulmonary administration after exposure to a pathogen to treat or inhibit the early infection of the respiratory tract.

27. (canceled)

28. The method according to claim 2, wherein the liposomal composition is administered two or three times per week.

29. The method according to claim 2, wherein the subject is selected from the group consisting of humans of all ages, primates, cynomolgus monkeys, rhesus monkeys mammals cattle, pigs, horses, sheep, goats, cats, dogs, and birds or
the subject is a human or
the subject is an individual with compromised/reduced immunity in the airways, or
the subject has a past history of smoking or is a current smoker.

30.-33. (canceled)

34. A device for nasal administration comprising the liposomal composition according to claim 2.
